(19) United States
(12) Patent Application Publication
(10) Pub. No.: US 2008/0176303 A1
(43) Pub. Date: Jul. 24, 2008

(54) Farm Scale Ethanol Plant

(75) Inventor: Cecil T. Massie, Bloomington, MN (US)

Correspondence Address: BROMBERG & SUNSTEIN LLP, 125 SUMMER STREET, BOSTON, MA, 02110-1618, US

(73) Assignee: 6Solutions, LLC, Bloomington, MN (US)

(21) Appl. No.: 12/016,784

(22) Filed: Jan. 18, 2008

Related U.S. Application Data
(60) Provisional application No. 60/881,386, filed on Jan. 19, 2007.

(51) Int. Cl. C12P 7/08

(52) U.S. Cl. 435/163

(57) ABSTRACT

An energy-efficient cycle for the production of ethanol and optionally natural gas from sunlight and biomass comprising first producing or obtaining biomass, for example by planting and harvesting of either an annual or perennial crop or both, and optionally tilling, fertilizing and obtaining wood wastes or paper wastes on-site or from a secondary site, for example, to supplement the on-site production of biomass, or for use as the sole source of biomass. The biomass is then converted to fermentable sugars through enzymatic conversion, acid hydrolysis, thermal cracking, or other processes that reduce cellulosic and hemi-cellulosic compounds to simple sugars. The reduced product is then fermented, producing ethanol in an intermediate beer product followed by recovery of ethanol from the resulting fermentation beer, wherein liquids are concentrated and recovered as fuel grade product. Recovery of ethanol may be by any suitable means including distillation, membrane separation or similar methods, and chromatographic rectification

### CROSS REFERENCE TO RELATED APPLICATIONS

This application claims priority from U.S. Provisional Patent Application Ser. No. 60/881,386, filed Jan. 19, 2007, the entire contents of which are incorporated by reference herein.

### TECHNICAL FIELD

The present invention relates to production of fuel grade ethanol, specifically design and operation of smaller scale—“farm scale”—ethanol plants for producing fuel grade ethanol in a distributed manner to facilitate the recovery and reuse of non-fuel components for crop production. The process described in this invention may be applied at any scale but is most advantageously applied at farm scale where the economics of nutrient recovery and recycle are greatest.

### BACKGROUND ART

The production of fuel grade ethanol is a major industry in the United States, projected to produce 5 billion gallons of ethanol by 2008. Under current technology, fuel grade ethanol is produced primarily from corn along with a co-product spent grain and soluble product commonly known as DDGS—dried distillers grain and solubles.

Corn production is an energy and nutrient intense crop. It is common practice to fertilize with anhydrous ammonia at rates of 160 or 170 pounds of elemental nitrogen, 60 to 70 pounds per acre of P₂O₅ (phosphorus), and 50 pounds per acres of K₂O (potassium).

While the overall energy balance of fossil fuel inputs to ethanol fuel outputs has raged for decades, little has been made of the continuing demand for N, P and K, which are largely drained from corn producing regions. While these elements are not destroyed and are largely contained in the distillers grains, the sale of the distillers grains co-product moves vast quantities of these nutrients from the regions where they are needed for crop production into regions where there is not agronomic uptake in crops of these nutrients, and so they accumulate in soil, pollute ground water, and threaten the long term viability of not only the corn belt but also the regions where the processing and feeding out of the distillers grains is done.

The production of ammonia for corn fertilizer is a major energy consumer in the corn-fuel cycle. Recycling nitrogen from corn back to fertilizers would significantly increase the net renewable energy output of fuel ethanol production.

Renewable energy is inherently tied to agriculture and forestry because the process of photosynthesis is the predominate mechanism for collecting and storing solar energy. In the end, energy is stored primarily in the form of carbon-carbon and carbon-hydrogen bonds. The other elements, N, P, K and water are not part of the energy transport mechanism.

The process for converting corn to ethanol is water intensive and wasteful. It takes 3 to 4 gallons of make up water, typically fresh water from surface water or wells, to produce a single gallon of ethanol under current technology. Owing to the need to transport dried grains long distances and store them for long periods of time, the primary water discharge from an ethanol plant is via the DDGS dryer exhaust. It is included in the intent of this invention to recover and recycle the water contained in the co-products back into crop production or the process.

A further consideration is that the basic fermentation/distillation process for fuel ethanol production is energy intensive. Ideally, the process will contain within its own feedstock and co-products the energy necessary for the process.

Finally, the process must be operable with minimum labor input and production must be coordinated over the entire collection of related facilities. For this function the proposed process will use a layered process control in which oversight and monitoring is done remotely from a centralized location. This permits the process to operate essentially unattended during most of the production cycle.

To achieve these objectives, what is clearly needed is a process optimized around sustainability in which only energy-bearing components leave the farm while crop nutrients and water are recycled to crop production.

### SUMMARY OF THE INVENTION

In a first embodiment of the invention there is provided a process of converting solar energy from biomass into ethanol and recovering and recycling nutrients and water from the process at a single geographic site, the process comprising obtaining biomass from the geographical site, converting the biomass to fermentable sugars, fermenting the fermentable sugars to produce an intermediate beer product, recovering ethanol from the beer product, leaving a residual stream of digester feed product, also referred to as dried distiller's grains and solubles, and water, and recovering water from the residual stream to obtain digester feed product, recovering nutrients from the digester feed product, using at least some of the recovered water in at least one of the converting and fermenting processes, and recycling at least some of the recovered nutrients at the geographic site, wherein the obtaining, converting, fermenting, recovering, using and recycling steps can all occur at the single geographical site.

The process so described may further comprise recycling the digester feed product by composting the feed product, recycling the digester feed product by using the feed product as fertilizer, digesting at least some of the residual stream to produce biogas and digester discharge, and/or deriving fuel from the biogas and using the fuel to power recovering ethanol from the beer products.

Related embodiments provide a process as described, wherein recovering nutrients further comprises sending the digester feed product to a storage tank and concentrating nutrients. More particularly, concentrating nutrients further comprises growing algae in the storage tank or precipitating ammonia, phosphorous and magnesium in the form of struvite.

Other related embodiments further comprise using the algae as a source of biomass and/or using the struvite as a fertilizer, particularly as a slow-release organic fertilizer.

The biomass may be obtained by planting and harvesting either an annual or perennial crop or both, and may also comprise tilling, fertilizing, and additionally obtaining wood wastes or paper wastes on-site, or from a secondary site. When the wood wastes or paper wastes are obtained on site, or from a secondary site, those may be used as the sole source of biomass.

In related embodiments, the annual or perennial crop or both may include corn, wheat, milo, oats, soybeans, hay, alfalfa, timber, sugar cane, algae and combinations thereof.

### BRIEF DESCRIPTION OF THE DRAWINGS

The foregoing features of the invention will be more readily understood by reference to the following detailed description, taken with reference to the accompanying drawings, in which:

FIG. 1 Co-Production of Ethanol and Natural Gas

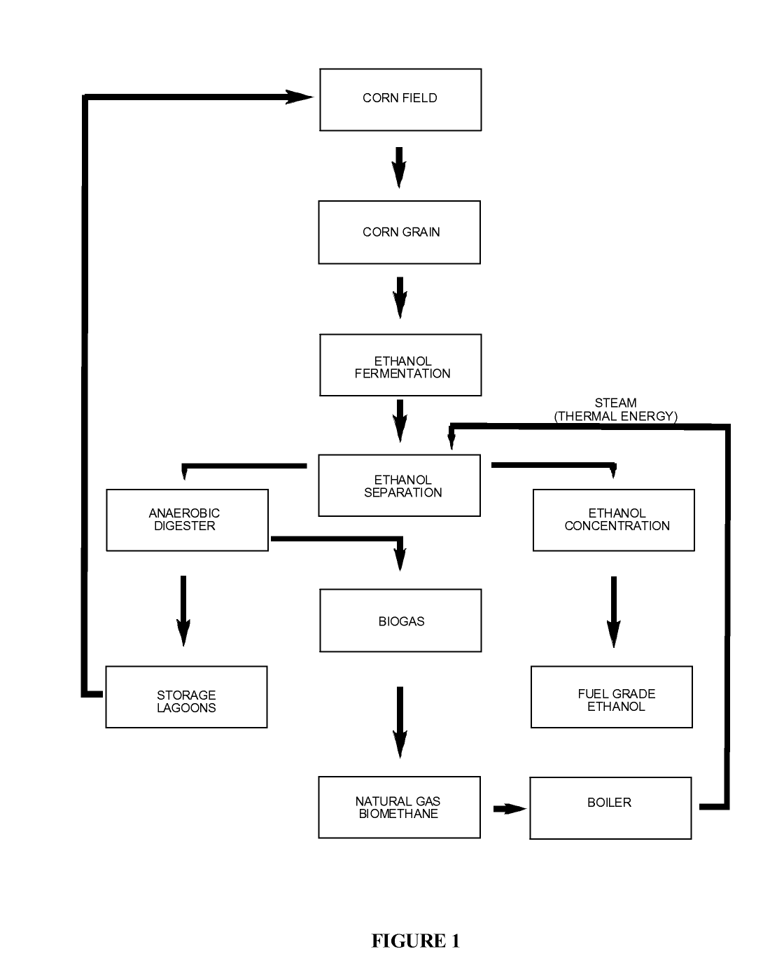

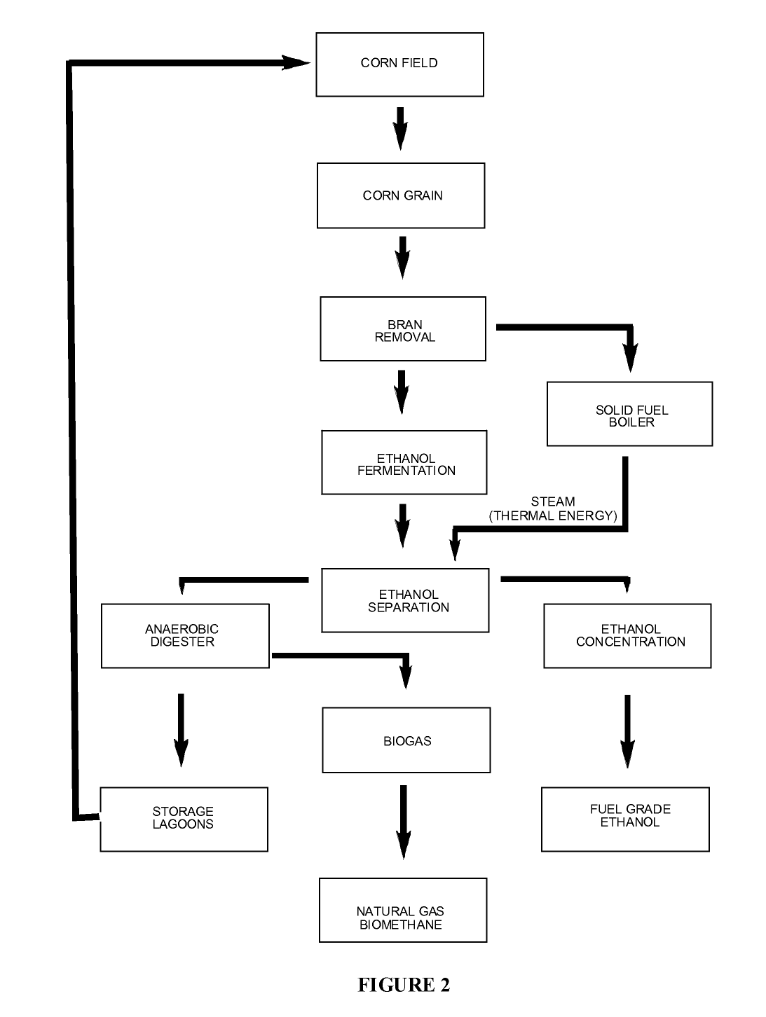

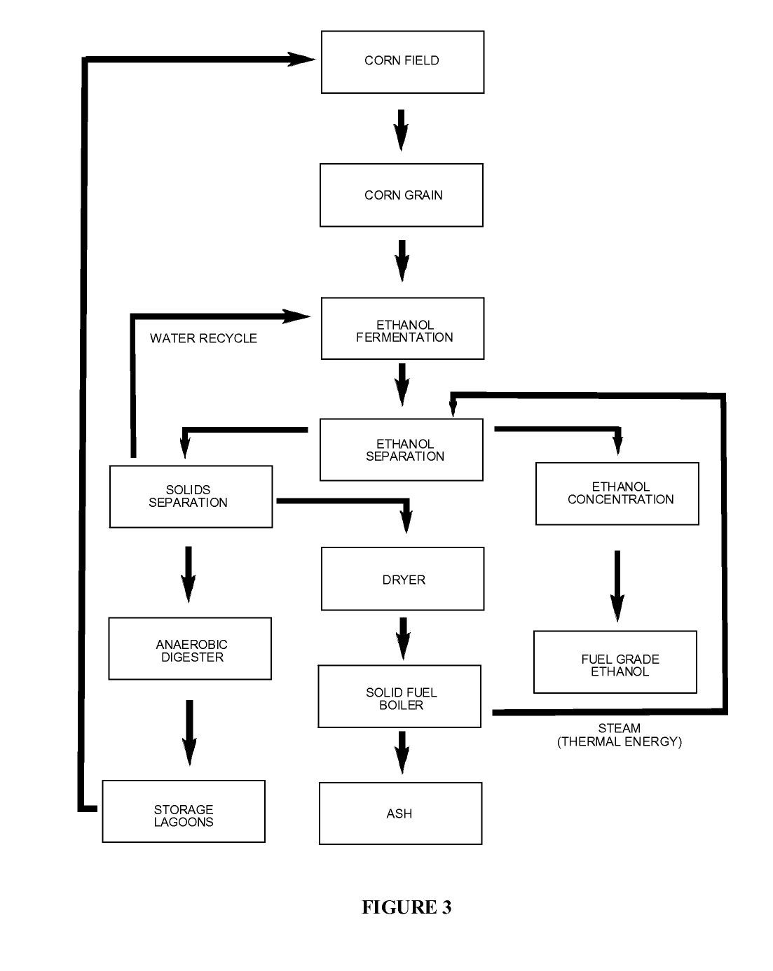

As shown in FIG. 1, energy production begins with production of corn grain by conventional cultivation and the corn is converted to ethanol by conventional fermentation. Fuel ethanol is recovered from the resulting beer by conventional distillation technology or optionally by the chromatic rectification process detailed in pending U.S. patent application Ser. No. Ser. No. 11/471,460 filed Jun. 20, 2006, the contents of which are hereby incorporated by reference herein.

The residual water and solids recovered from the bottom of the distillation process is directed to an anaerobic digester in which the residual solids are converted to biogas, a mixture of carbon dioxide and methane.

A portion of the biogas either before or after refining may be directed to a boiler to produce steam for use in the ethanol process. The rest of the biogas may be refined to pipeline natural gas by selective stripping of the carbon dioxide using amines or water. Biomethane produced in this manner may be shipped as compressed natural gas or injected into the natural gas distribution pipeline system.

In a further refinement of the natural gas production aspect of this invention, biogas from multiple digesters may be collected using underground pipelines and delivered to a central refining facility adjacent to a natural gas main.

In a further refinement, ethanol produced in a distributed manner may be piped through dedicated underground pipelines to a central storage and load-out facility for national distribution by truck or rail.

The residual water fraction after the anaerobic digester is directed to pond storage where it is retained under cover to preserve the nitrogen content until applied to the fields in the next growing season. Soluble potassium and phosphorous fractions are retained in the water and are available as fertilizer upon application to the land.

FIG. 2: Distributed Ethanol Production with Pre-fractionation of the corn

As shown in FIG. 2. an alternate application of the present invention envisions a partial fractionation of the corn before the fermentation to separate a portion of the corn bran from the remainder of the corn grain. This separated bran fraction may then be burned in conventional solid fuel burners to produce steam for the process. Ash from the burning process is gathered together with the water fraction produced by anaerobic digestion as before and land applied in the next crop season.

FIG. 3: Distributed Ethanol Production with wet bran combustion

As shown in FIG. 3. an alternate application of the present invention envisions separation of all or a portion of the suspended solids from the stillage are separated from the water fraction and burned to produce steam for the ethanol process. Optionally the separated solids may be partially or substantially dried to improve combustion characteristics. As in FIG. 2, the ash from the combustion is combined with the anaerobic digester discharge and recycled to the corn fields.

### DETAILED DESCRIPTION OF SPECIFIC EMBODIMENTS

Definitions. As used in this description and the accompanying claims, the following terms shall have the meanings indicated, unless the context otherwise requires:

“Biomass” means, in the context of this application any lignocellulosic material such as corn stalks, wood, straw or similar material.

“Mash” means, in the context of this application, a fermentable starchy mixture such as a mixture of milled grain or other fermentable carbohydrate in water, which is used in the production of ethanol. “Mash” may be a mixture of boiled grain, bran or other substances (including bacteria) for brewing or production purposes and it may also be fed to livestock and fowl. The term may be used at any stage from the initial mixing of the feedstock in water, prior to any cooking and saccharification, through to the completion of fermentation, when it becomes referred to as “beer.”

“Beer” means, in the context of this application, any cellulose, hemi-cellulose or fermentable sugar source (such as grains) that has been allowed to ferment into an ethanol-containing aqueous mixture, and includes ethanol from recovery of gums.

“Farm-scale” means, in the context of this application, fermentation/distillation/chromatographic rectification technology that is feasible for use on a farm, farm being defined as any land used for the production of crops and/or livestock having approximately 5 or more acres.

“Fuel grade ethanol” means, in the context of this application, ethanol produced from biomass by fermentation containing from 50% water to as little as 0.5% or less water or other impurities, as desired, according to need or interest.

### Overview of Distributed Ethanol Production

Distributed ethanol production begins with on-farm fractionation of biomass into a carbon-hydrogen rich fraction which is comprised of ethanol and water at ethanol concentrations from 50 to 99.5% by volume, into a methane-rich biogas fraction, and into a nutrient rich water faction containing essentially all of the N,P, and K in the original biomass supply.

There have been attempts in the past to add value to corn by processing it to ethanol. These attempts, once described as “A Still on Every Hill” ¹ universally failed to achieve financial success². It was a characteristic of these earlier efforts that the distillers grain co-product was to be fed to livestock and the waste water or stillage was to be used as drinking water for the livestock. Functionally the livestock replaced the dryer and evaporator in the conventional process. ¹ A movement in the U.S. in the 1970s to put an ethanol still on every farm for farmers to add value to their operations by producing their own ethanol for fuel.² See “Will Backyard Stills Make a Comeback” by Anduin Kirkbride McElroy, EthanolProducer Magazine, July 1006. (This is Google's cache of http://www.ethanolproducer.com/article.jsp?article_id=2154 as retrieved on Aug. 16, 2006 14:10:28 GMT.)

Attempts to scale up this concept to large scale ethanol plants feeding dairy herds or feedlot operations have been proposed of late. In some of these operations, anaerobic digestion of the animal wastes has been added to produce biogas to use either in the ethanol process or for the production of electricity.

But all of these efforts fail on two key points:

1. For the most part, the animals are not where the corn or biomass is, and the nutrient surplus resulting from large-scale feeding operations is not addressed; and

2. Even when a balance can be struck, there are simply not enough animals in the country to absorb the volume of co-product that will be produced from the massive quantities of ethanol needed to have meaningful effect on the nation's transportation and liquid fuels supply.

On-Farm ethanol production has historically been disadvantaged by the relative economics of scale, and the ability of large operations to purchase corn on relatively advantageous terms. The more modest farm operations, however, for example farms of approximately 2,000 acres or so, given the proper circumstances, have the potential to gain a competitive advantage over even very large operations by vertical integration of crop nutrient production within an on-Farm ethanol plant.

### EXAMPLES

In one specific embodiment there is provided an energy-efficient cycle for the production of ethanol and optionally natural gas from sunlight and biomass comprising first producing or obtaining biomass, for example by planting and harvesting of either an annual or perennial crop or both, which crops may include, but are not limited to, corn, wheat, milo, oats, soybeans, hay, alfalfa, timber, sugar cane, algae and combinations thereof. If necessary tilling and fertilizing may also be performed as part of the obtaining of biomass. Other ways for obtaining biomass may include obtaining wood wastes or paper wastes on-site or from a secondary site, for example, to supplement the on-site production of biomass, or for use as the sole source of biomass.

After obtaining the biomass, it is converted to fermentable sugars through enzymatic conversion, acid hydrolysis, thermal cracking, or other processes that reduce cellulosic and hemi-cellulosic compounds to simple sugars. The simple sugars are then fermented to ethanol to produce an intermediate beer product followed by recovery of ethanol from the resulting fermentation beer. As shown in FIGS. 1-3, the biomass is corn grain, which is subjected to fermentation. The fermented ethanol is then separated into solids and liquids. The liquids are then concentrated and recovered as fuel grade product. Recovery of ethanol may be by any suitable means including distillation, membrane separation or similar methods, and chromatographic rectification such as detailed in pending U.S. patent application Ser. No. 11/471,460 filed Jun. 20, 2006, such that the ethanol is substantially separated from the remaining stillage (residual stream) of the fermentation beer, leaving in particular the elemental components nitrogen, phosphorus, potassium and sulfur in the residual stream.

Solids—i.e. wet grains—in the residual stream may optionally be separated by screening, centrifugation or settling, followed by recycling a portion of the recovered water stream to the front of the process for use in preparing the fermentation mash. After ethanol separation, as shown in FIGS. 1-3, the residual stream may also be optionally passed through an anaerobic digester to produce biogas, comprised primarily of carbon dioxide and methane, as detailed in FIGS. 1-3, or first passed through an additional solids separation step, as depicted in FIG. 3 before going to the anaerobic digester. Optionally, the resulting biogas/biomethane may be further collected and processed to yield pipeline-quality natural gas, as shown in FIGS. 1 and 2, or may be, whether as biomethane or pipeline-quality natural gas, the energy supply for the ethanol process. A further option is to use either compressed biogas or biomethane to fuel the tractors and other farm equipment.

In addition, as shown in FIGS. 1-3, the discharge from the anaerobic digester may be optionally sent to storage lagoons and then recovered for land application, such as application to a corn field, in the manner of liquid manure by truck transport and/or pipeline to irrigation booms. In one particular embodiment, as seen in FIG. 3, the solids may be separated before the anaerobic digester step, with some sent to a dryer to be used for fuel in a solid fuel boiler to produce ash, and other solids proceeding to the anaerobic digester and then on to the storage lagoons. The ash produced from solids sent to the boiler are then added to the discharge from the anaerobic digester in the storage lagoons, and recovered for land application, as described above.

Further, the residual stillage stream, whether with or without digestion, may be optionally blended onto a compostible solid such that the blended mixture has nutrient and water content to facilitate bacterial decomposition of the blend, the compostible solid optionally including the wet grains recovered from the stillage, as described above, or optionally being other compostible material such as sawdust, corn stalks, hay or similar crop or biomass residuals as well as recovered paper, pulp or similar materials and manure from animal or human wastes.

The compostible solids are composted either by placement in rotating compost devices or by spreading the liquid fraction on wind rows of biomass, followed by recovery of composted residual mulch for storage and land application during the next cycle of crop production.

In such a process, energy from the sun and water, used to grow crops that become the biomass for the fermentation process, are recycled during the fermentation process through production of biogas, recovery of solids and water from the residual stream, wherein the fuel is used to drive the fermentation process and the recovered solids and water are used for compositing and fertilization, and for irrigation and use in the fermentation process, respectively. Once the process begins, the cycle is essentially a steady-state process where energy in equals energy out. Moreover, the nutrients, depleted from the soil during production of the biomass crops, get returned to the soil through the recycling of the recovered solids, thus minimizing the loss of K, N and P from the geographical location where they are continually needed, and concomitantly reducing the build-up and pollution of these un-needed elements at geographical locations where they are not needed.

### Further Refinements

Further energy efficient enhancements may be added to the process to make it entirely self-sufficient on energy. For example, the energy for mash preparation and distillation may come from combusting biogas produced in the anaerobic digester; the digester feed may be heated to digester temperature and the digester maintained at temperature by burning biogas; the digester may be heated to digestion temperature and maintained at temperature by recovery of heat from the composting stop; the digester may be heated to digestion temperature and maintained at temperature by recovery of heat from the fermentation step; and/or the water produced and vented by the composting step may be optionally condensed and recycled to the fermentation process thereby reducing the net use of water in the overall process.

In addition, to return nutrients back to the fields, the lagoon storage step may include growing algae as a source of biomass or as a method to concentrate nutrients for recycling them to the field. The algae growth extracts nutrients from the water phase and allows a larger portion of the process water to be short circuited back to the process rather than held and applied to the corn field.

As an alternative, ammonia, phosphorous and magnesium may be precipitated from the lagoon water in the form of struvite, a slow release, organic fertilizer.

Both options make it possible for the nutrients to be returned to the fields in a more dense form, and for the system as a whole to use less water, and for more water to be recycled back into the process.

### Improvements

The proposed process is a material improvement over conventional technology and meets a long recognized and un-met need for sustainable production of ethanol and/or natural gas from crops and timber. The process requires minimal outside input of nutrients and energy. This is beneficial to the farmer because the profits of manufacturing and distributing these nutrients and supplies is now incorporated into the profitability of the farming operation itself. The process is also sustainable, recycling all non-carbon elements over the range of the farm itself. For example, Nitrogen leaving composting is converted into an organic form, which fixes the nitrogen in place, preventing loss to waterways or the air. Moreover, the harvest of a portion of the corn stalks is beneficial to carbon sequestration in the soil because it promotes no-till farming, which has been show to increase soil carbon content.

In addition, the process is capital/cost efficient, requiring minimal investment in facilities not directly related to ethanol production. By contrast, in a conventional ethanol plant design, nearly one-half of the total capital investment is in equipment to recover, dry and handle DDGS co-product. Finally, the process can be practiced as organic farming.

The above embodiments are not intended to be inclusive, and other embodiments may be envisioned that satisfy the elements of on-farm ethanol production and nutrient and water recovery and recycling that fall within the scope of the present technology and invention.

What is claimed is:

1. A process of converting solar energy from biomass into ethanol and recovering and recycling nutrients and water from the process at a single geographic site, the process comprising:
obtaining biomass from the geographical site;
converting the biomass to fermentable sugars;
fermenting the fermentable sugars to produce an intermediate beer product;
recovering ethanol from the beer product, leaving a residual stream of digester feed product and water; and
recovering water from the residual stream to obtain the digester feed product;
recovering nutrients from the digester feed product;
using at least some of the recovered water in at least one of the converting and fermenting processes; and
recycling at least some of the recovered nutrients at the geographic site, wherein the obtaining, converting, fermenting, recovering, using and recycling steps can all occur at the single geographical site.

2. A process according to claim 1, further comprising:
recycling the digester feed product by composting the feed product.

3. A process according to claim 1, further comprising:
recycling the digester feed product by using the feed product as fertilizer.

4. A process according to claim 1, wherein recovering nutrients further comprises sending the digester feed product to a storage tank and concentrating nutrients.

5. A process according to claim 4, wherein concentrating further comprises growing algae in the storage tank.

6. A process according to claim 4, wherein concentrating further comprises precipitating ammonia, phosphorous and magnesium in the form of struvite.

7. A process according to claim 5, further comprising using the algae as a source of biomass.

8. A process according to claim 6, further comprising using the struvite as a slow release organic fertilizer.

9. A process according to claim 1, further comprising:
digesting at least some of the residual stream to produce biogas and digester discharge.

10. A process according to claim 1, further comprising:
deriving fuel from the biogas and using the fuel to power recovering ethanol from the beer products.

11. A process according to claim 1, wherein obtaining biomass comprises:
planting and harvesting of a crop.

12. A process according to claim 11, wherein the crop is an annual crop.

13. A process according to claim 11, wherein the crop is a perennial crop.

14. A process according to claim 11, wherein the crop is both an annual crop and a perennial crop.

15. A process according to claim 1, wherein obtaining further comprises:
additionally obtaining wood and/or paper wastes.

16. A process according to claim 15 wherein the wastes are obtained on-site,

17. A process according to claim 15 wherein the wastes are obtained from a secondary site.

18. A process according to claim 15, further comprising using the wastes as the sole source of biomass.

19. A process according to claim 1 1, wherein the crop comprises any one of corn, wheat, milo, oats, soybeans, hay, alfalfa, timber, sugar cane algae and combinations thereof.

20. A process according to claim 14, wherein the crop comprises any one of corn, wheat, milo, oats, soybeans, hay, alfalfa, timber, sugar cane, algae and combinations thereof.
